(19) United States
(12) Patent Application Publication
(10) Pub. No.: US 2026/0020975 A1
(43) Pub. Date: Jan. 22, 2026

(54) ORAL APPLIANCE

(71) Applicant: DCSTAR INC, New York, NY (US)

(72) Inventor: David LUO, New York, NY (US)

(73) Assignee: DCSTAR INC, New York, NY (US)

(21) Appl. No.: 18/777,015

(22) Filed: Jul. 18, 2024

(51) Int. Cl. A61F 5/56

(52) U.S. Cl. CPC A61F 5/566; A61F 2005/563

(57) ABSTRACT

An oral appliance, which is a convenient, cost-effective, and comfortable oral appliance for users, and includes an upper tray assembly and a lower tray assembly. The upper tray assembly includes an upper moldable component and an upper frame, while the lower tray assembly includes a lower moldable component and a lower frame. The upper and lower moldable components are made from a moldable material that can deform when heated. The upper and lower frames do not deform when heated to the temperature required for deforming the upper and lower moldable components. The upper moldable component is configured to be detachably connected to the upper frame, and the lower moldable component is configured to be detachably connected to the lower frame.

### TECHNICAL FIELD

The present disclosure relates to oral appliances, particularly to an appliance for preventing, reducing, or eliminating snoring and/or obstructive sleep apnea.

### BACKGROUND

Obstructive sleep apnea (OSA) is a sleep-related breathing disorder caused by the relaxation of muscles in the tongue, soft palate, and the area behind the oropharynx during sleep. This condition typically leads to breathing cessation episodes lasting 10 seconds or longer, occurring 5-30 times or more per hour. Persistent OSA results in excessive daytime sleepiness, nighttime snoring, morning headaches, and may cause cardiovascular diseases and brain damage, among other complications.

Currently, there are various methods to prevent snoring and/or obstructive sleep apnea. One method involves advancing the lower jaw relative to the upper jaw to increase airflow through the throat passage. Since the 19th century, it has been proved that advancing the lower jaw can enhance airflow through the throat passage, thereby reducing or eliminating snoring and/or obstructive sleep apnea.

Mandibular advancement devices (MADs) in oral appliances are configured to adjust the user's lower jaw position to help alleviate or prevent snoring and sleep apnea. Typically, the principle of MADs is to position the lower jaw (mandible) forward of its neutral position to increase airflow through the throat passage. Thus, oral appliances aid in reducing or eliminating snoring and/or obstructive sleep apnea.

Although oral appliances help reduce or eliminate snoring and/or obstructive sleep apnea, customizing such devices by dental care professionals requires additional work and frequent visits to the dental clinic for fitting or adjustments, increasing the user's time and monetary costs. Furthermore, when users, particularly individuals, attempt to self-fit the MADs, they often face issues like discomfort.

### SUMMARY

Based on the above deficiencies, the present disclosure provides an oral appliance that is user-friendly, reduces the user's cost of use, and is comfortable to wear.

The disclosure discussed herein provides an oral appliance, which includes: an upper tray assembly configured to be positioned within a user's oral cavity adjacent to the user's upper dental arch, and a lower tray assembly configured to be positioned within the user's oral cavity adjacent to the user's lower dental arch. The upper tray assembly includes an upper moldable component and an upper frame, while the lower tray assembly includes a lower moldable component and a lower frame. In addition, the upper moldable component and the lower moldable component include a moldable material that can deform when heated, and the upper frame and the lower frame do not deform when heated to a temperature required for deforming the upper moldable component and the lower moldable component. The upper moldable component is configured to be detachably connectable to the upper frame, whereas the lower moldable component is configured to be detachably connectable to the lower frame. The upper moldable component and the lower moldable component each have at least one outer wall, and the upper frame and the lower frame have at least one inner wall.

The outer wall of the upper moldable component and the outer wall of the lower moldable component are each at least partially in contact with the at least one inner wall of the upper frame and the at least one inner wall of the lower frame, respectively.

In an embodiment, a shape of the upper tray assembly is configured to substantially conform to a curvature of the user's upper dental arch.

In an embodiment, a shape of the lower tray assembly is configured to substantially conform to a curvature of the user's lower dental arch.

In an embodiment, the upper moldable component and the lower moldable component include a flexible thermoplastic material.

In an embodiment, the upper frame and the lower frame at least partially include a material that is relatively more rigid compared to the upper moldable component and the lower moldable component.

This disclosure also provides another oral appliance. The oral appliance includesan upper tray assembly configured to be positioned within a user's oral cavity adjacent to the user's upper dental arch, and a lower tray assembly configured to be positioned within the user's oral cavity adjacent to the user's lower dental arch. The upper tray assembly includes an upper moldable component and an upper frame, while the lower tray assembly includes a lower moldable component and a lower frame. The upper moldable component and the lower moldable component include a moldable material that can deform when heated, and the upper frame and the lower frame do not deform when heated to a temperature required for deforming the upper moldable component and the lower moldable component. The upper moldable component is configured to be detachably connectable to the upper frame, whereas the lower moldable component is configured to be detachably connectable to the lower frame. In addition, a projected area of the upper moldable component on a horizontal plane is greater than a projected area of the upper frame on the same plane, and a projected area of the lower moldable component on a horizontal plane is greater than a projected area of the lower frame on the same plane.

In an embodiment, the temperature required for deforming the modable material of the upper moldable component and the lower moldable component is less than 100 degrees Celsius.

In an embodiment, the upper frame and the lower frame include a material with high temperature stability, and a temperature required for deforming the upper frame and the lower frame is greater than 100 degrees Celsius.

In an embodiment, a ratio of the projected area of the upper moldable component and the lower moldable component to the projected area of the upper frame and the lower frame on a horizontal plane is greater than 1.

In an embodiment, a length of ends extending outward from the upper moldable component and the lower moldable component is greater than a length of ends extending outward from the upper frame and the lower frame.

This disclosure provides yet another oral appliance, which includes an upper tray assembly configured to be positioned within a user's oral cavity adjacent to the user's upper dental arch, and a lower tray assembly configured to be positioned within the user's oral cavity adjacent to the user's lower dental arch. The upper tray assembly includes an upper moldable component and an upper frame, while the lower tray assembly includes a lower moldable component and a lower frame. The upper tray assembly and/or the lower tray assembly includes a connection part configured to connect the upper moldable component and the lower moldable component to the upper frame and the lower frame, respectively. In addition, the upper moldable component and the lower moldable component include a moldable material that can deform when heated, and the upper frame and the lower frame do not deform when heated to a temperature required for deforming the upper moldable component and the lower moldable component. The upper moldable component is configured to be detachably connectable to the upper frame, whereas the lower moldable component is configured to be detachably connectable to the lower frame.

In an embodiment, the upper frame and the lower frame are detachably connectable to the upper moldable component and the lower moldable component respectively, via additional parts.

In an embodiment, the connection part includes protrusions, slots, and/or additional parts that include bolt latches, screws, pins, or magnets.

In an embodiment, the protrusions are in a shape of at least one of cylindrical, square, spherical, T-shaped, L-shaped, or other shapes.

In an embodiment, the protrusions are provided on the upper moldable component and the lower moldable component and match with the slots on the upper frame and the lower frame, respectively.

In an embodiment, the slots are provided on the upper moldable component and the lower moldable component and match with the protrusions on the upper frame and the lower frame, respectively.

The disclosure further provides an oral appliance, which includes an upper tray assembly configured to be positioned within a user's oral cavity adjacent to the user's upper dental arch, and a lower tray assembly configured to be positioned within the user's oral cavity adjacent to the user's lower dental arch. The upper tray assembly includes an upper moldable component and an upper frame, while the lower tray assembly includes a lower moldable component and a lower frame. The upper moldable component and the lower moldable component include a moldable material that can deform when heated, and the upper frame and the lower frame do not deform when heated to a temperature required for deforming the upper moldable component and the lower moldable component. The upper moldable component is configured to be detachably connectable to the upper frame, whereas the lower moldable component is configured to be detachably connectable to the lower frame. The upper frame and/or the lower frame have at least one slot.

In an embodiment, the at least one slot in the upper frame and the lower frame is configured to accommodate overflowing material of the upper moldable component and the lower moldable component during the thermoforming process.

In an embodiment, a shape of the upper moldable component and the lower moldable component is at least one of ovoid arch, tapered arch, or square arch, to conform to a curvature of the user's dental arch.

In an embodiment, the upper frame and the lower frame have at least one inner wall and at least one outer wall, and a distance between the at least one inner wall and the at least one outer wall is at least partially between 0.3 to 8 mm.

The oral appliance of the present disclosure offers at least the following beneficial effects:
1. As the moldable components are configured to be detachably connected to the frames, and the process of detachment and installation is simple and straightforward, users can quickly and conveniently replace the moldable components without needing to replace the entire device. This enhances the flexibility and maintainability of the device. Additionally, since only parts of the device need to be replaced rather than the whole device, this design significantly reduces material waste, contributing to lower resource consumption. This demonstrates notable environmental and climate friendliness. By reducing waste generation and promoting resource reuse, the disclosure helps lower carbon emissions and environmental impact.
2. The moldable components are made from a moldable material that can deform when heated. Users can place the heated moldable components into their oral cavity and shape them to fit their dental arch by biting down. If any errors occur during the operation and biting process, or if the bite is not properly aligned, users can easily replace the moldable components and re-bite due to the detachable connection design of the moldable components and the frames. There is no need to replace the entire device. This design not only significantly reduces resource and monetary waste but also enhances the adjustability of the device and improves user experience.
3. The moldable components include flexible thermoplastic material and are configured to withstand the user's biting force, which means their lifespan is typically shorter than that of the frames. When the moldable components are damaged or need replacement, only the moldable components need to be replaced due to their detachable connection with the frames, without replacing the entire device. Additionally, if the frames need to be replaced while the moldable components are still usable, the pre-shaped moldable components can be mounted onto the new frames without the need for reshaping. This design saves time, effectively reduces resource and monetary waste, and significantly improves the device's economic efficiency and usability. By reducing material consumption and extending the lifespan of each component, it helps lower carbon emissions and environmental impact.
4. The frames are provided with slots that not only secure the protrusions of the moldable components but also accommodate the overflowing material during the thermoforming process. The overflowing material fills the slots upon cooling, further enhancing the fixation between the frames and the moldable components. This disclosure achieves double fixation of the moldable components through the slot structure in the frames, improving connection stability and simplifying the installation process, ensuring the device's reliability during use. Moreover, the simple structure of the connection parts between the frames and the moldable components allows for easy detachment of the moldable components from the frames, facilitating maintenance and replacement, and achieving the device's detachable functionality.
5. Compared to the frames, the moldable components use flexible materials, which better adapt to the shape of the oral cavity, reducing irritation and abrasion to the soft tissues. Additionally, the horizontal projected area of the moldable components is larger than that of the frames, which helps reduce contact pressure on the soft tissues, further minimizing the risk of abrasions. Considering the varying distances from the inner lip to the posterior molar region among different users, the design of the oral appliance allows users to trim the length to fit this distance. The soft moldable components enable users to easily trim and adjust the device length according to personal needs, enhancing comfort and personalized adaptation. It is noteworthy that since the horizontal projected area of the moldable components is larger than that of the frames, trimming does not affect the structure and function of the frames, ensuring the stability and effectiveness of the oral appliance.

### BRIEF DESCRIPTION OF THE DRAWINGS

FIG. 1 is an overall schematic diagram of an embodiment of an oral appliance according to an embodiment of the present disclosure;

FIG. 2 is an exploded view schematic diagram of the structure of an oral appliance according to several embodiments of the present disclosure;

FIGS. 3A, 3B, and 3C are top views showing the moldable components and the shapes of the dental arch according to several embodiments of the present disclosure;

FIG. 4 is a schematic diagram of the horizontal plane, sagittal plane, and coronal plane according to several embodiments of the present disclosure;

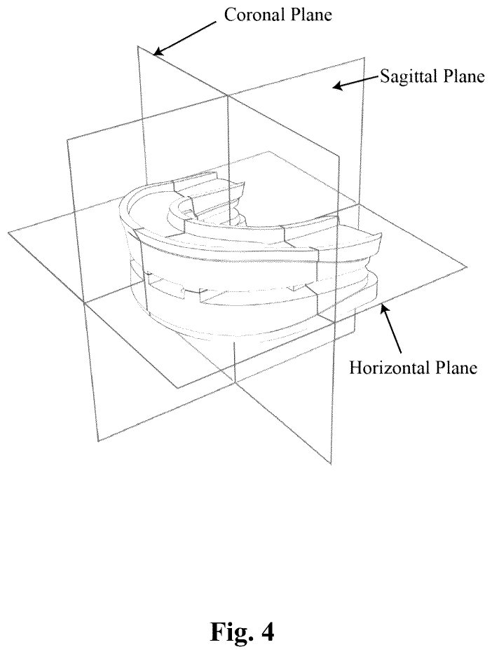

FIG. 5 is a top view of the upper tray assembly according to several embodiments of the present disclosure;

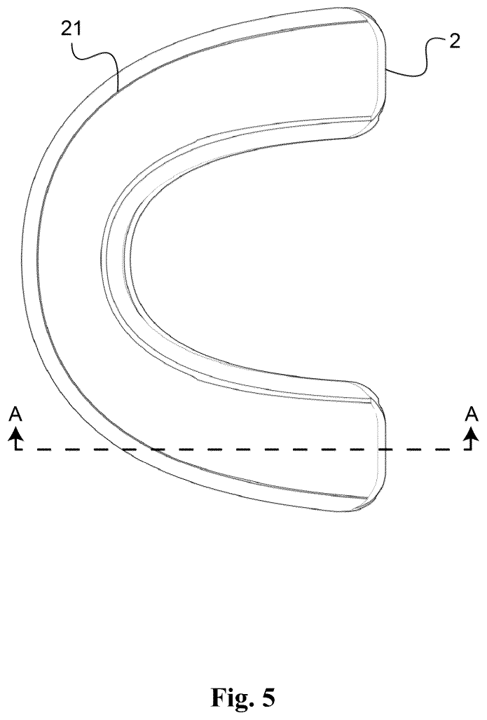

FIG. 6 is a cross-sectional schematic diagram of the connection part according to an embodiment of the present disclosure;

FIG. 7 is a structural schematic diagram of the lower tray assembly according to an embodiment of the present disclosure;

FIG. 8 is an exploded view schematic diagram of the upper moldable component and the upper frame according to an embodiment of the present disclosure;

FIG. 9 is a cross-sectional schematic diagram of the upper moldable component and the upper frame according to an embodiment of the present disclosure;

FIG. 10 is an exploded view schematic diagram of the upper moldable component and the upper frame according to an embodiment of the present disclosure;

FIG. 11 is an exploded view schematic diagram of the upper moldable component and the upper frame according to an embodiment of the present disclosure;

FIG. 12 is a cross-sectional schematic diagram of the ends of the upper moldable component and the upper frame according to an embodiment of the present disclosure;

FIG. 13 is a partial comparative schematic diagram of the connection part of the upper moldable component and the upper frame before and after thermoforming according to an embodiment of the present disclosure.

### DETAILED DESCRIPTION

To make the objectives, features, and advantages of the present disclosure more apparent and understandable, detailed descriptions of specific embodiments of the disclosure will be provided below in conjunction with the accompanying drawings. Numerous specific details are set forth in the following description to provide a thorough understanding of the disclosure. However, the disclosure can be practiced in various ways other than those described herein, and those skilled in the art can make similar modifications without departing from the spirit of the disclosure. Therefore, the disclosure is not limited to the specific embodiments disclosed below.

The present disclosure relates to an oral appliance 1, particularly to a device for preventing, reducing, or eliminating snoring and/or obstructive sleep apnea. In certain embodiments, the appliance can also function as a sports mouthguard for the upper dental arch, lower dental arch, or both dental arches. In some embodiments, the appliance can also serve as a bruxism guard acting on the upper dental arch, lower dental arch, or both dental arches.

The disclosure provides a convenient, cost-effective, and comfortable oral appliance 1 for users.

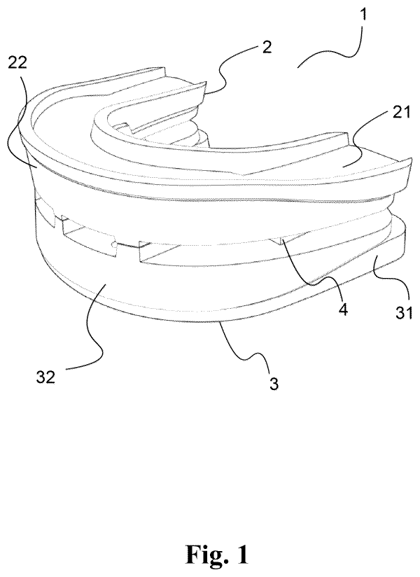

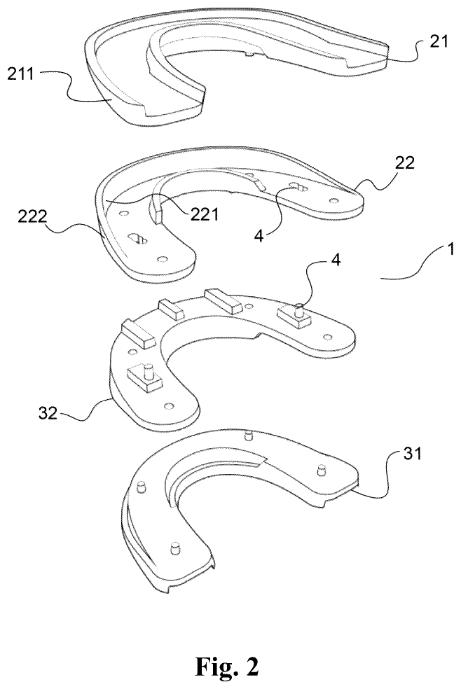

Specifically, with reference to FIGS. 1 to 3A, 3B, and 3C, the oral appliance 1 of the present disclosure includes an upper tray assembly 2 and a lower tray assembly 3. The upper tray assembly 2 is configured to be positioned within the user's oral cavity adjacent to the user's upper dental arch. The lower tray assembly 3 is configured to be positioned within the user's oral cavity adjacent to the user's lower dental arch. Furthermore, when positioned within the user's oral cavity, the upper tray assembly 2 is located below the user's upper dental arch, and the lower tray assembly 3 is located above the user's lower dental arch. The dental arch typically presents an arched structure that conforms to the natural alignment of the user's teeth. Specifically, both the upper dental arch and the lower dental arch form a substantially symmetrical and approximately arcuate curvature. The shapes of the upper tray assembly 2 and the lower tray assembly 3 are configured to substantially conform to the curvature of the user's dental arches, providing a better fit for the user's upper and lower teeth, thus offering sufficient support and stability for the oral appliance 1.

The upper tray assembly 2 includes an upper moldable component 21 and an upper frame 22, while the lower tray assembly 3 includes a lower moldable component 31 and a lower frame 32. In this disclosure, the frames 22 and 32 include the upper frame 22 and the lower frame 32, and the moldable components 21 and 31 include the upper moldable component 21 and the lower moldable component 31. The materials for the upper tray assembly 2 and the lower tray assembly 3 can be broadly selected from a range of plastics or polymer materials suitable for oral medical devices and capable of being molded. These materials include, but are not limited to, polypropylene (PP), polycarbonate (PC), polyethylene terephthalate (PET), polymethyl methacrylate (PMMA), polyphenylsulfone (PPSU), polyethylene (PE), high-density polyethylene (HDPE), low-density polyethylene (LDPE), ethylene-vinyl acetate copolymer (EVA), thermoplastic polyurethane (TPU), styrene-ethylene-butylene-styrene (SEBS), styrene-ethylene-propylene-styrene (SEPS), and silicone rubber. These materials exhibit good biocompatibility, mechanical properties, and processing performance, which are advantageous for the manufacturing of the oral appliance 1. The various parts of the upper tray assembly 2 and the lower tray assembly 3, such as the moldable components 21 and 31 and the frames 22 and 32, can be made from a single material or a combination of multiple materials. This multi-material design can fully utilize the characteristics of different materials to meet various needs during the use of the oral appliance 1. A multi-layer structure can also be adopted, where each layer may include the same or different materials, allowing for optimized material properties. The polymer materials may contain various fillers, plasticizers, pigments, and other additives to meet different performance requirements and manufacturing needs. For example, nanoscale fillers can be added to enhance the strength and hardness of the material, or antimicrobial agents can be added to improve the hygiene performance of the oral appliance. Suitable types of polymer materials include thermoplastics, thermosetting polymers, elastomers, and thermoplastic elastomers. These materials can be copolymers, terpolymers, or mixtures of different types of polymers, such as mixtures of thermoplastics with different molecular weights. These materials possess good processing and mechanical properties, which are beneficial for the manufacturing of the oral appliance 1.

The upper tray assembly 2 and/or the lower tray assembly 3 also include a fixation part 4, which is configured to connect and/or secure the upper tray assembly 2 and the lower tray assembly 3. When the user uses the oral appliance, the fixation part 4 positions the user's lower jaw forward relative to the upper jaw, achieving the function of mandibular advancement.

The material selection for the fixation part 4 should ensure a tight connection between the tray assemblies 2 and 3 to guarantee the stability and reliability of the oral appliance 1. The fixation part 4 can include the same moldable or polymer materials as the upper tray assembly 2 and lower tray assembly 3, or other materials suitable for oral medical devices and appliances, such as metals. The fixation part 4 can be composed of a single material or a combination of multiple materials.

The moldable components 21 and 31 are made from a moldable material, preferably a thermoplastic, allowing users to heat and shape them for adaptive adjustments. The heating and shaping method used in this disclosure is called “boil—and—bite,” a simple and practical technique where the user heats the oral appliance 1 in hot water and then bites it to mold it to their teeth for a snug fit. The moldable components 21 and 31 can deform when heated, while the frames 22 and 32 do not deform at the temperature required for the moldable components 21 and 31 to deform.

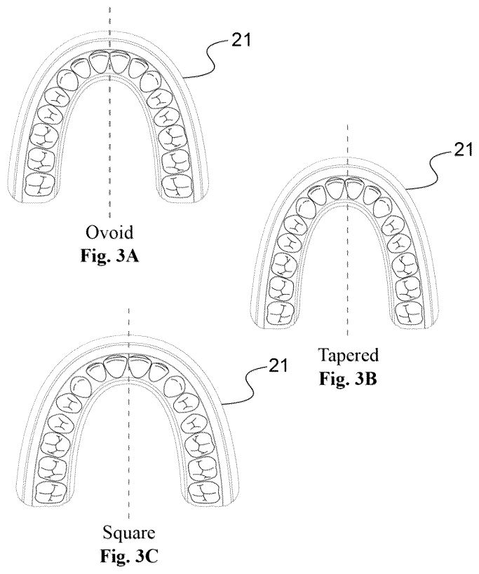

The moldable components 21 and 31 are made from a moldable material, and a temperature required for deforming the moldable components 21 and 31 is less than 100 degrees Celsius, while the frames 22 and 32 are made from a material with high-temperature stability, and a temperature required for deforming the frames 22 and 32 is greater than 100 degrees Celsius. This design ensures the stability and reliability of the oral appliance 1 during the shaping process, allowing the user to easily perform adaptive adjustments for a better user experience. If the frames 22 and 32 also include thermoplastic materials, their deformation temperature will be higher than that of the moldable components 21 and 31, ensuring that the frames 22 and 32 are not affected during the molding process of the moldable components 21 and 31. Moreover, as shown in FIGS. 3A, 3B, and 3C, the shapes of the user's dental arch can generally be categorized into ovoid arch, tapered arch, square arch, and other shapes. Therefore, the moldable components 21 and 31 can also adopt at least one of these shapes-ovoid arch, tapered arch, square arch—to conform to the user's dental arch curvature.

The moldable components 21 and 31 include flexible thermoplastic material, which is sufficiently soft and possesses good elasticity and plasticity. These components can deform when heated and retain their shape upon cooling. This allows the moldable components 21 and 31 to better conform to the shape and structure of the user's upper and lower teeth, providing a better fit and enhanced comfort. The upper moldable component 21 is configured to fit the user's upper dental arch, while the lower moldable component 31 is configured to fit the user's lower dental arch. Made from moldable materials, the upper moldable component 21 can deform when heated to adapt and adjust to the user's upper jaw. Similarly, the lower moldable component 31 can deform when heated to adapt and adjust to the user's lower jaw. This design ensures that the oral appliance 1 accurately covers and fits the user's teeth surfaces, providing better adaptability and comfort.

Compared to the moldable components 21 and 31, the frames 22 and 32 at least partially include a relatively more rigid material with a hardness range between Shore D45 to Shore D95. The frames 22 and 32 have at least one inner wall 221 and at least one outer wall 222, with the distance between the at least one inner wall 221 and the at least one outer wall 222 being at least partially between 0.3 to 8 mm. This is to provide the necessary stability and support for the oral appliance 1. The rigid design of the frames 22 and 32 ensures that the oral appliance 1 does not easily deform or loosen during use, maintaining its stability and functionality. This design preserves the overall structural stability of the oral appliance 1 while providing sufficient support, allowing the user to comfortably use the oral appliance 1.

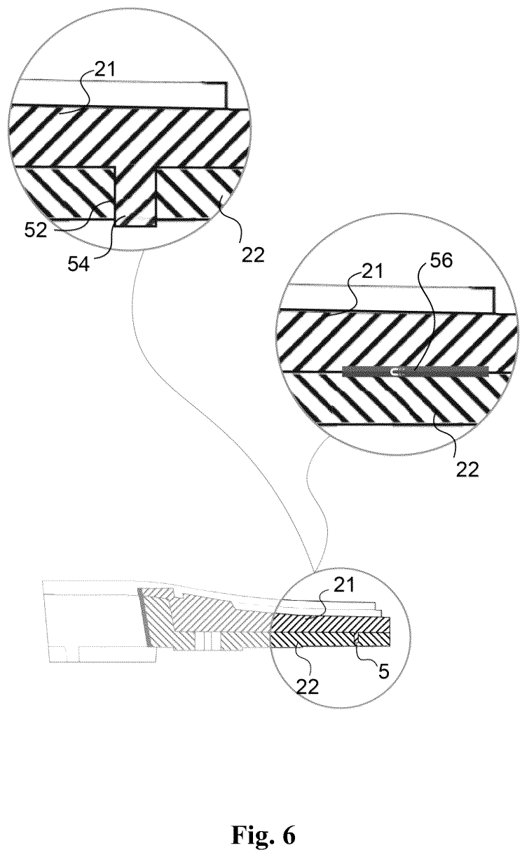

The moldable components 21 and 31 are configured to be detachably connected to the frames 22 and 32. As shown in FIG. 6, the upper tray assembly 2 and/or the lower tray assembly 3 have a connection part 5. The connection part 5 is configured to connect the moldable components 21 and 31 with the frames 22 and 32. The connection part 5 includes protrusions 54 and 55, slots 52 and 53 (see FIG. 6), and/or additional components 56 that include bolt latches, screws, pins, or magnets (see FIG. 6). This means that the frames 22 and 32 can be detachably connected to the moldable components 21 and 31 via snaps and/or additional parts. In this disclosure, the detachable connection design of the moldable components 21 and 31 with the frames 22 and 32 is based on several considerations. Firstly, since the moldable components 21 and 31 are detachably connected to the frames 22 and 32, and the process of detaching and installing them is simple and straightforward, users can quickly and conveniently replace the moldable components 21 and 31 without replacing the entire device. This enhances the flexibility and maintainability of the device. Additionally, because only parts of the device need to be replaced rather than the whole device, this design significantly reduces material waste and helps lower resource consumption, demonstrating notable environmental and climate friendliness. By reducing waste generation and promoting resource reuse, the disclosure helps lower carbon emissions and environmental impact.

Secondly, the moldable components 21 and 31 are made from moldable materials that can deform when heated. This allows users to heat and shape the components themselves to achieve adaptive adjustments. Users simply need to place the upper tray assembly 2 and the lower tray assembly 3 in hot water to heat them, and then gently bite the upper tray assembly 2 and the lower tray assembly 3 with their mouth. This forms a good fit between the tray assemblies and the teeth, molding the heated moldable components 21 and 31 to conform to the dental arch. If there are any errors or improper bites during the process, the user can easily replace the moldable components 21 and 31 and re-bite, without needing to replace the entire device. This design not only significantly reduces resource and monetary waste but also enhances the adjustability of the device and improves user experience.

Finally, the moldable components 21 and 31 include flexible thermoplastic material and are designed to withstand the user's biting force, which typically results in a shorter lifespan compared to the frames 22 and 32. When the moldable components 21 and 31 are damaged or need to be replaced, they can be easily detached from the frames 22 and 32, requiring only the replacement of the moldable components and not the entire device. Additionally, if the frames 22 and 32 need replacement while the moldable components 21 and 31 are still usable, the shaped moldable components 21 and 31 can be mounted onto the new frames 22 and 32 without the need for reshaping. This design not only saves time but also effectively reduces resource and monetary waste, significantly improving the economic efficiency and usability of the device. By reducing material consumption and extending the lifespan of each component, this design helps lower carbon emissions and environmental impact.

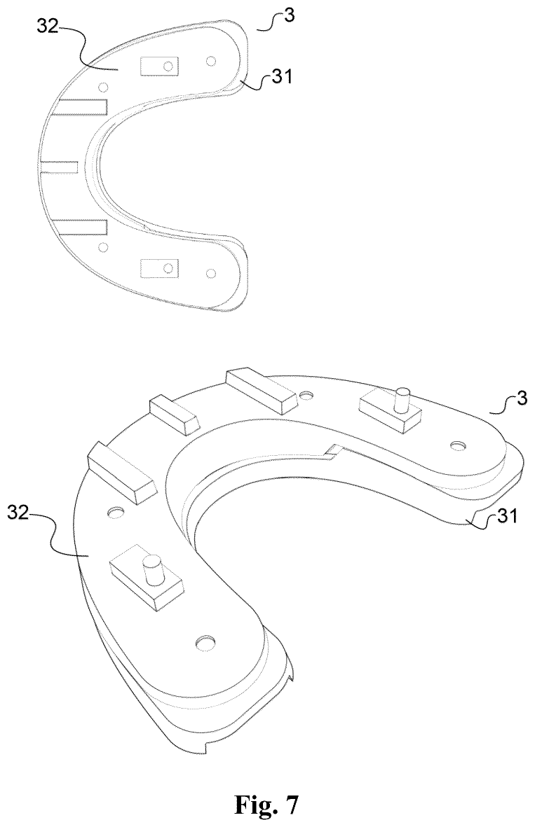

As shown in FIG. 7, the horizontal projected area of the moldable components 21 and 31 is greater than that of the frames 22 and 32, meaning the ratio of the horizontal projected area of the moldable components 21 and 31 to that of the frames 22 and 32 is greater than 1, providing greater comfort and stability. Specifically, the horizontal projected area of the frames 22 and 32 does not exceed 60 square centimeters, and a length of the ends extending outward from the moldable components 21 and 31 is greater than a length of the ends extending outward from the frames 22 and 32. The larger horizontal projected area of the moldable components 21 and 31 compared to the frames 22 and 32 is primarily for the following reasons: Firstly, the flexible material of the moldable components 21 and 31 better adapts to the shape of the oral cavity, reducing irritation and abrasion to the soft tissues. Secondly, the larger projected area of the moldable components 21 and 31 reduces contact pressure on the soft tissues, further minimizing the risk of abrasions. Furthermore, considering the variations in the distance from the inner lip to the posterior molar region among different users, overly long devices may cause abrasions to the posterior oral tissues, particularly in the joint area, and improper fitting devices may affect occlusal function. The oral appliance 1 in this embodiment allows users to trim the length to fit the distance from the inner lip to the posterior molar region, with the soft moldable components 21 and 31 enabling easy trimming and adjustment according to individual needs, thereby enhancing comfort and personalized adaptability. It is important to note that since the horizontal projected area of the moldable components 21 and 31 is larger than that of the frames 22 and 32, trimming thus does not affect the frames 22 and 32, ensuring the stability of the oral appliance 1. This design not only ensures the functionality of the oral appliance 1 but also gives users more control over adjustments, making the use of the oral appliance 1 more comfortable.

The following embodiments illustrate several structures of the oral appliance 1 according to the present disclosure.

### Embodiment 1

This embodiment of the oral appliance 1 includes: an upper tray assembly 2 configured to be positioned within a user's oral cavity adjacent to the user's upper dental arch; a lower tray assembly 3 configured to be positioned within the user's oral cavity adjacent to the user's lower dental arch. The upper tray assembly 2 includes an upper moldable component 21 and an upper frame 22; the lower tray assembly 3 includes a lower moldable component 31 and a lower frame 32. In this embodiment, the frames 22 and 32 include the upper frame 22 and the lower frame 32, and the moldable components 21 and 31 include the upper moldable component 21 and the lower moldable component 31.

The moldable components 21 and 31 include moldable materials that can deform when heated, while the frames 22 and 32 do not deform at the temperature required for the moldable components 21 and 31 to deform. The moldable components 21 and 31 are configured to be detachably connected to the frames 22 and 32. The upper tray assembly 2 and/or the lower tray assembly 3 include a connection part 5, which is configured to connect the moldable components 21 and 31 with the frames 22 and 32. The connection part 5 includes protrusions 54 and 55 and slots 52 and 53.

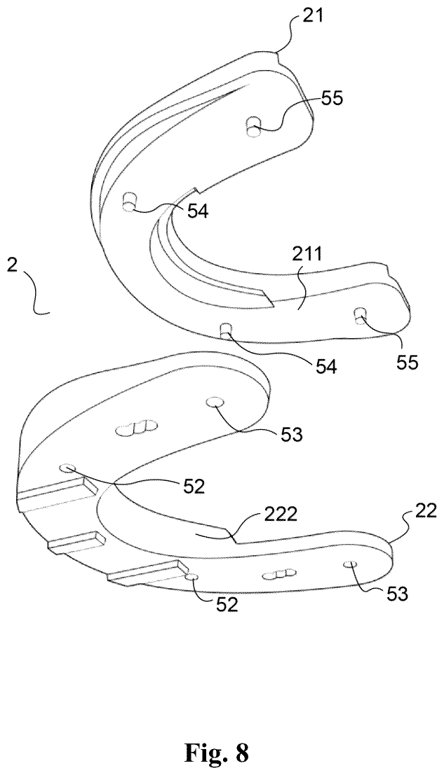

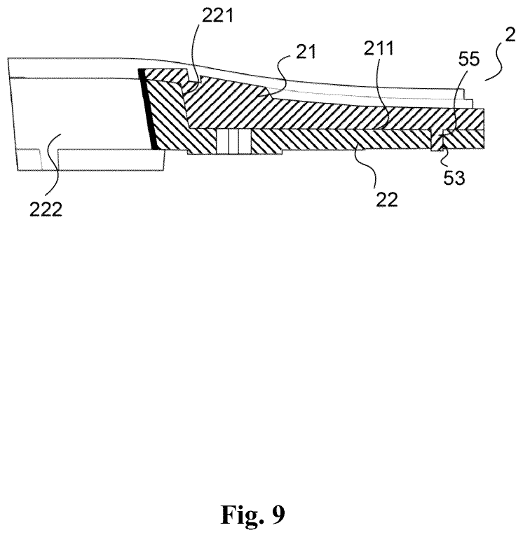

On the same tray assembly 2 or 3, the moldable components 21 and 31 have at least one protrusion 54 and 55, while the frames 22 and 32 have at least one slot 52 and 53. For better fixation, it is preferable that the moldable components 21 and 31 and the frames 22 and 32 each have four protrusions 54 and 55 and four slots 52 and 53, respectively. These are symmetrically distributed on both sides near the middle part (i.e., the part of the oral appliance close to the user's front teeth) and at both ends (i.e., the ends of the oral appliance close to the user's temporomandibular joints). As shown in FIGS. 8 and 9, taking the upper tray assembly 2 as an example, the upper moldable component 21 has four protrusions 54 and 55, with two protrusions 54 located on both sides near the middle part of the upper moldable component 21, and the other two protrusions 55 located at both ends of the upper moldable component 21. The upper frame 22 has four slots 52 and 53 corresponding to protrusions 54 and 55.

The protrusions 54 and 55 are located on the moldable components 21 and 31 and are configured to match the slots 52 and 53 on the frames 22 and 32. The protrusions 54 and 55 are integrally formed with the moldable components 21 and 31 and are shaped for easy production, installation, and detachment; i.e., the shapes of the protrusions 54 and 55 can be cylindrical, square, spherical, T-shaped, L-shaped, or at least one of other shapes. The frames 22 and 32 include at least one inner wall 221 and one outer wall 222, with the at least one inner wall 221 of the frame forming a cavity to accommodate the moldable components 21 and 31. During installation, the moldable components 21 and 31 are placed onto the frames 22 and 32 such that the outer wall 211 of the moldable components 21 and 31 are at least partially in contact with the at least one inner wall 221 of the frames 22 and 32. The protrusions 54 and 55 on the moldable components 21 and 31 are then inserted into the corresponding slots 52 and 53 on the frames 22 and 32. The slots 52 and 53 on the frames 22 and 32 have sufficient depth, ranging from 0.2 to 20 mm, to form a secure connection with the protrusions 54 and 55 on the moldable components 21 and 31.

### Embodiment 2

This embodiment of the oral appliance 1 includes: an upper tray assembly 2 configured to be positioned within a user's oral cavity adjacent to the user's upper dental arch; a lower tray assembly 3 configured to be positioned within the user's oral cavity adjacent to the user's lower dental arch. The upper tray assembly 2 includes an upper moldable component 21 and an upper frame 22; the lower tray assembly 3 includes a lower moldable component 31 and a lower frame 32. In this embodiment, the frames 22 and 32 include the upper frame 22 and the lower frame 32, and the moldable components 21 and 31 include the upper moldable component 21 and the lower moldable component 31.

The moldable components 21 and 31 include moldable materials that can deform when heated, while the frames 22 and 32 do not deform at the temperature required for the moldable components 21 and 31 to deform. The moldable components 21 and 31 are configured to be detachably connected to the frames 22 and 32. The upper tray assembly 2 and/or the lower tray assembly 3 include a connection part 5, which is configured to connect the moldable components 21 and 31 with the frames 22 and 32. The connection part 5 includes protrusions 54 and 55 and slots 52 and 53.

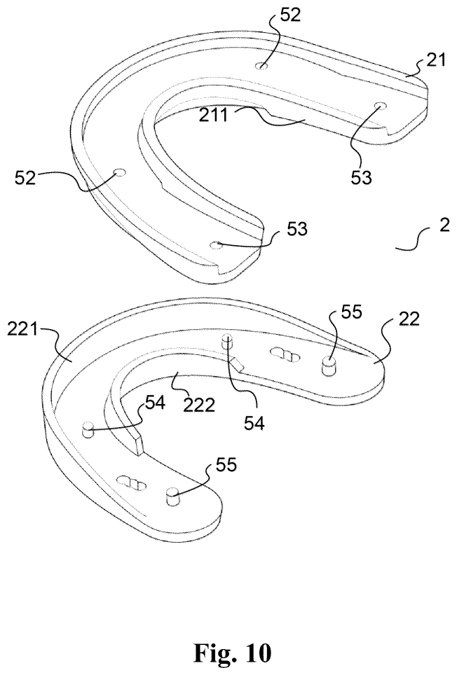

The difference between this embodiment and Embodiment 1 is that the protrusions and slots of the moldable components 21 and 31 and the frames 22 and 32 are opposite each other, but they function the same way. The slots 52 and 53 are located on the moldable components 21 and 31 and match the protrusions 54 and 55 on the frames 22 and 32. The frames 22 and 32 include at least one inner wall 221 and one outer wall 222, with the at least one inner wall 221 forming a cavity to accommodate the moldable components 21 and 31. The slots 52 and 53 are located on the moldable components 21 and 31 and correspond in shape to the protrusions 54 and 55. For better fixation, it is preferable that the frames 22 and 23 and the moldable components 21 and 31 each have four protrusions 54 and 55 and four slots 52 and 53. These are symmetrically distributed on both sides near the middle part (i.e., the part of the oral appliance close to the user's front teeth) and at both ends (i.e., the ends of the oral appliance close to the user's temporomandibular joints). Taking the upper tray assembly 2 as an example, as shown in FIG. 10, the upper moldable component 21 has four slots 52 and 53, with two slots 52 located on both sides near the middle part of the upper moldable component 21, and the other two slots 53 located at both ends of the upper moldable component 21.

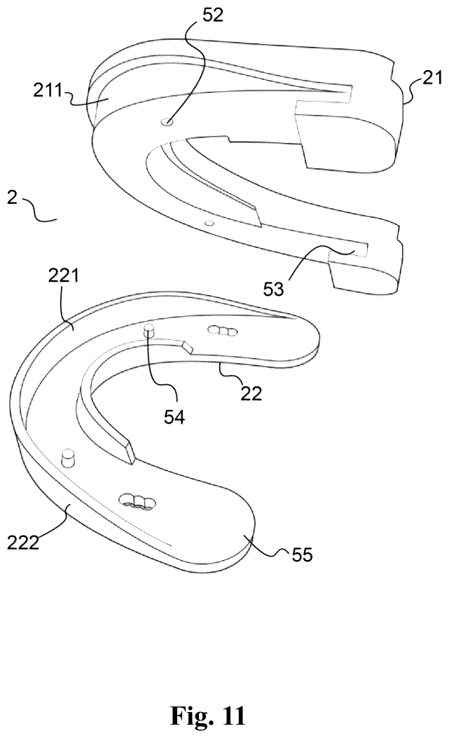

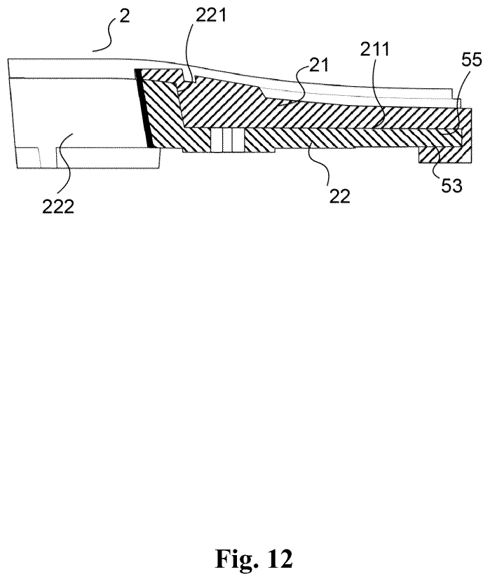

In other embodiments, the slots 53 at both ends of the moldable components 21 and 31 extend to enclose the ends of the frames 22 and 32. As shown in FIGS. 11 and 12, the moldable components 21 and 31 extend partially beyond the ends of the frames 22 and 32 and then extend back in the opposite direction to form hollow slots 53 that enclose the protrusions 55 of the frames 22 and 32. This structure makes the ends of the moldable components 21 and 31 longer than the ends of the frames 22 and 32, preventing the more rigid ends of the frames 22 and 32 from contacting the user's posterior molar region. Instead, the softer moldable components 21 and 31 come into contact, better adapting to the shape of the oral cavity and reducing irritation and abrasion to the posterior molar region of the user.

### Embodiment 3

This embodiment of the oral appliance 1 includes: an upper tray assembly 2 configured to be positioned within a user's oral cavity adjacent to the user's upper dental arch; and a lower tray assembly 3 configured to be positioned within the user's oral cavity adjacent to the user's lower dental arch. The upper tray assembly 2 includes an upper moldable component 21 and an upper frame 22; the lower tray assembly 3 includes a lower moldable component 31 and a lower frame 32. In this embodiment, the frames 22 and 32 include the upper frame 22 and the lower frame 32, and the moldable components 21 and 31 include the upper moldable component 21 and the lower moldable component 31.

The moldable components 21 and 31 include moldable materials that can deform when heated, while the frames 22 and 32 do not deform at the temperature required for the moldable components 21 and 31 to deform. The moldable components 21 and 31 are configured to be detachably connected to the frames 22 and 32. The upper tray assembly 2 and/or the lower tray assembly 3 include a connection part 5, which is configured to connect the moldable components 21 and 31 with the frames 22 and 32. The connection part 5 includes protrusions 54 and 55 and slots 51, 52, and 53.

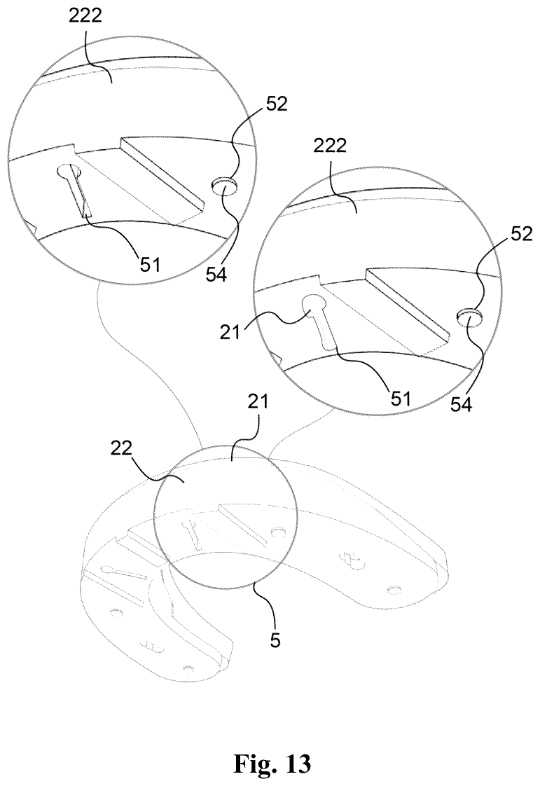

In this embodiment, the moldable components 21 and 31 each have at least one protrusion 54 and 55, while the frames 22 and 32 have slots 51, 52, and 53. The protrusions 54 and 55 on the moldable components 21 and 31 correspond to the slots 52 and 53 on the frames 22 and 32. Additionally, the frames 22 and 32 include at least one inner wall 221, one outer wall 222, and slots 51. The distance between the inner wall 221 and the outer wall 222 is at least partially 0.3 to 8 mm. When the user heats the oral appliance 1 in hot water and bites down gently, the moldable components 21 and 31 deform due to the bite, causing excess material to overflow from the sides. This can result in protrusions on the surface of the oral appliance 1, causing discomfort and affecting its appearance. Therefore, in this embodiment, slots 51 are provided on the frames 22 and 32 to accommodate the overflowing material of the moldable components 21 and 31 during the thermoforming process. As shown in FIG. 13, the slots 51 penetrate the inner wall 221 and outer wall 222 of the frames 22 and 32 (as seen in FIG. 13). When the moldable components 21 and 31 are thermoformed, the excess material overflows into the slots 51. Due to the varying structures and material properties of the moldable components 21 and 31, sufficient slots must be provided to accommodate the overflowing material. The horizontal area of slots 51 is at least 3 square millimeters. When the moldable components 21 and 31 cool down, the overflowing material in the slots 51 solidifies, forming protrusions (as seen in FIG. 13), further enhancing the fixation between the frames 22 and 32 and the moldable components 21 and 31. Additionally, the technical features in the above embodiments can be combined as needed to obtain an oral appliance 1 that includes all or some of the mentioned technical features.

The oral appliance of the present disclosure offers at least the following beneficial effects:
1. As the moldable components are configured to be detachably connected to the frames, and the process of detachment and installation is simple and straightforward, users can quickly and conveniently replace the moldable components without needing to replace the entire device. This enhances the flexibility and maintainability of the device. Additionally, since only parts of the device need to be replaced rather than the whole device, this design significantly reduces material waste, contributing to lower resource consumption. This demonstrates notable environmental and climate friendliness. By reducing waste generation and promoting resource reuse, the disclosure helps lower carbon emissions and environmental impact.
2. The moldable components are made from a moldable material that can deform when heated. Users can place the heated moldable components into their oral cavity and shape them to fit their dental arch by biting down. If any errors occur during the operation and biting process, or if the bite is not properly aligned, users can easily replace the moldable components and re-bite due to the detachable connection design of the moldable components and the frames. There is no need to replace the entire device. This design not only significantly reduces resource and monetary waste but also enhances the adjustability of the device and improves user experience.
3. The moldable components include flexible thermoplastic material and are configured to withstand the user's biting force, which means their lifespan is typically shorter than that of the frames. When the moldable components are damaged or need replacement, only the moldable components need to be replaced due to their detachable connection with the frames, without replacing the entire device. Additionally, if the frames need to be replaced while the moldable components are still usable, the pre-shaped moldable components can be mounted onto the new frames without the need for reshaping. This design saves time, effectively reduces resource and monetary waste, and significantly improves the device's economic efficiency and usability. By reducing material consumption and extending the lifespan of each component, it helps lower carbon emissions and environmental impact.
4. The frames are provided with slots that not only secure the protrusions of the moldable components but also accommodate the overflowing material during the thermoforming process. The overflowing material fills the slots upon cooling, further enhancing the fixation between the frames and the moldable components. This disclosure achieves double fixation of the moldable components through the slot structure in the frames, improving connection stability and simplifying the installation process, ensuring the device's reliability during use. Moreover, the simple structure of the connection parts between the frames and the moldable components allows for easy detachment of the moldable components from the frames, facilitating maintenance and replacement, and achieving the device's detachable functionality.
5. Compared to the frames, the moldable components use flexible materials, which better adapt to the shape of the oral cavity, reducing irritation and abrasion to the soft tissues. Additionally, the horizontal projected area of the moldable components is larger than that of the frames, which helps reduce contact pressure on the soft tissues, further minimizing the risk of abrasions. Considering the varying distances from the inner lip to the posterior molar region among different users, the design of the oral appliance allows users to trim the length to fit this distance. The soft moldable components enable users to easily trim and adjust the device length according to personal needs, enhancing comfort and personalized adaptation. It is noteworthy that since the horizontal projected area of the moldable components is larger than that of the frames, trimming does not affect the structure and function of the frames, ensuring the stability and effectiveness of the oral appliance.

The technical features of the above-described embodiments can be combined in any manner. For the sake of brevity, not all possible combinations of the various technical features in the above embodiments are described. However, as long as there is no contradiction in these combinations of technical features, they should be considered within the scope of the present disclosure. In addition, it must be noted that as used herein and in the appended claims, the regular forms “a” “an” “the” include their plural equivalents, unless the context clearly dictates otherwise.

The above-described embodiments only represent a few implementations of the present disclosure. Although the descriptions are relatively specific and detailed, they should not be interpreted as limiting the scope of the disclosure. It should be noted that those skilled in the art can make various modifications and improvements without departing from the concept of the disclosure, and these should all fall within the scope of the present disclosure. Therefore, the scope of protection for this disclosure should be determined by the appended claims.

1. An oral appliance, comprising:
an upper tray assembly configured to be positioned within a user's oral cavity adjacent to a user's upper dental arch;
a lower tray assembly configured to be positioned within the user's oral cavity adjacent to a user's lower dental arch;
wherein the upper tray assembly includes an upper moldable component and an upper frame stacked in a first direction;
wherein the lower tray assembly includes a lower moldable component and a lower frame stacked in the first direction;
wherein the upper moldable component and the lower moldable component include a moldable material that can deform when heated, and the upper frame and the lower frame do not deform when heated to a temperature required for deforming the upper moldable component and the lower moldable component;
wherein the upper moldable component is configured to be detachably connectable to an upper surface of the upper frame;
wherein the lower moldable component is configured to be detachably connectable to a lower surface of the lower frame;
wherein the upper moldable component and the lower moldable component each have at least one outer wall;
wherein the upper frame and the lower frame have at least one inner wall;
wherein the at least one outer wall of the upper moldable component and the at least one outer wall of the lower moldable component are each at least partially in contact with the at least one inner wall of the upper frame and the at least one inner wall of the lower frame, respectively,
wherein, during a shaping process, the upper moldable component is connected to the upper frame by protrusions formed by overflowing material of the upper moldable component when heated and then cooled,
wherein, during the shaping process, the lower moldable component is connected to the lower frame by protrusions formed by overflowing material of the lower moldable component when heated and then cooled,
wherein ends of the upper moldable component extend beyond ends of the upper frame in a second direction perpendicular to the first direction.

2. The oral appliance according to claim 1, wherein a shape of the upper tray assembly is configured to conform to a curvature of the user's upper dental arch.

3. The oral appliance according to claim 1, wherein a shape of the lower tray assembly is configured to conform to a curvature of the user's lower dental arch.

4. The oral appliance according to claim 1, wherein the upper moldable component and the lower moldable component include a flexible thermoplastic material.

5. The oral appliance according to claim 1, wherein the upper frame and the lower frame at least partially include a material that is relatively more rigid compared to the upper moldable component and the lower moldable component.

6. An oral appliance, comprising:
an upper tray assembly configured to be positioned within a user's oral cavity adjacent to a user's upper dental arch;
a lower tray assembly configured to be positioned within the user's oral cavity adjacent to a user's lower dental arch;
wherein the upper tray assembly includes an upper moldable component and an upper frame stacked in a first direction;
wherein the lower tray assembly includes a lower moldable component and a lower frame stacked in the first direction;
wherein the upper moldable component and the lower moldable component include a moldable material that can deform when heated, and the upper frame and the lower frame do not deform when heated to a temperature required for deforming the upper moldable component and the lower moldable component;
wherein the upper moldable component is configured to be detachably connectable to an upper surface of the upper frame;
wherein the lower moldable component is configured to be detachably connectable to a lower surface of the lower frame;
wherein a projected area of the upper moldable component on a horizontal plane is greater than a projected area of the upper frame on the same plane;
wherein a projected area of the lower moldable component on a horizontal plane is greater than a projected area of the lower frame on the same plane,
wherein, during a shaping process, the upper moldable component is connected to the upper frame by protrusions formed by overflowing material of the upper moldable component when heated and then cooled,
wherein, during the shaping process, the lower moldable component is connected to the lower frame by protrusions formed by overflowing material of the lower moldable component when heated and then cooled,
wherein ends of the lower moldable component extend beyond ends of the lower frame in a second direction perpendicular to the first direction.

7. The oral appliance according to claim 6, wherein the temperature required for deforming the moldable material of the upper moldable component and the lower moldable component is less than 100 degrees Celsius.

8. The oral appliance according to claim 6, wherein the upper frame and the lower frame include a material with high temperature stability, and a temperature required for deforming the upper frame and the lower frame is greater than 100 degrees Celsius.

9. The oral appliance according to claim 6, wherein a ratio of the projected area of the upper moldable component and the lower moldable component to the projected area of the upper frame and the lower frame on a horizontal plane is greater than 1.

10. (canceled)

11. An oral appliance, comprising:
an upper tray assembly configured to be positioned within a user's oral cavity adjacent to a user's upper dental arch;
a lower tray assembly configured to be positioned within the user's oral cavity adjacent to a user's lower dental arch;
wherein the upper tray assembly includes an upper moldable component and an upper frame stacked in a first direction;
wherein the lower tray assembly includes a lower moldable component and a lower frame stacked in the first direction;
wherein the upper tray assembly and/or the lower tray assembly includes a connection part configured to connect the upper moldable component and the lower moldable component to the upper frame and the lower frame, respectively;
wherein the upper moldable component and the lower moldable component include a moldable material that can deform when heated, and the upper frame and the lower frame do not deform when heated to a temperature required for deforming the upper moldable component and the lower moldable component;
wherein the upper moldable component is configured to be detachably connectable to an upper surface of the upper frame;
wherein the lower moldable component is configured to be detachably connectable to a lower surface of the lower frame,
wherein, during a shaping process, the connection part comprises protrusions formed by overflowing material of the upper moldable component and the lower moldable component when heated and then cooled,
wherein ends of the upper moldable component extend beyond ends of the upper frame in a second direction perpendicular to the first direction.

12. The oral appliance according to claim 11, wherein the upper frame and the lower frame are detachably connectable to the upper moldable component and the lower moldable component respectively, via additional parts.

13. The oral appliance of according to claim 11, wherein the connection part further includes pre-formed protrusions, slots, bolt latches, screws, pins, or magnets.

14. The oral appliance according to claim 13, wherein the protrusions are in a shape of at least one of cylindrical, square, spherical, T-shaped, or L-shaped.

15. The oral appliance according to claim 13, wherein the protrusions are provided on the upper moldable component and the lower moldable component and match with the slots on the upper frame and the lower frame, respectively.

16. The oral appliance according to claim 13, wherein the slots are provided on the upper moldable component and the lower moldable component and match with the protrusions on the upper frame and the lower frame, respectively.

17. An oral appliance, comprising:
an upper tray assembly configured to be positioned within a user's oral cavity adjacent to a user's upper dental arch;
a lower tray assembly configured to be positioned within the user's oral cavity adjacent to a user's lower dental arch;
wherein the upper tray assembly includes an upper moldable component and an upper frame stacked in a first direction;
wherein the lower tray assembly includes a lower moldable component and a lower frame stacked in a first direction;
wherein the upper moldable component and the lower moldable component include a moldable material that can deform when heated, and the upper frame and the lower frame do not deform when heated to a temperature required for deforming the upper moldable component and the lower moldable component;
wherein the upper moldable component is configured to be detachably connectable to an upper surface of the upper frame;
wherein the lower moldable component is configured to be detachably connectable to a lower surface of the lower frame;
wherein the upper frame and/or the lower frame have at least one slot,
wherein, during process, the upper moldable component is connected to the upper frame by protrusions formed by overflowing material of the upper moldable component when heated and then cooled,
wherein, during the shaping process, the lower moldable component is connected to the lower frame by protrusions formed by overflowing material of the lower moldable component when heated and then cooled, and
wherein ends of the upper moldable component extend beyond ends of the upper frame in a second direction perpendicular to the first direction.

18. The oral appliance of claim 17, wherein the at least one slot in the upper frame and the lower frame is configured to accommodate overflowing material of the upper moldable component and the lower moldable component, respectively, during a thermoforming process.

19. The oral appliance of claim 17, wherein the upper moldable component and the lower moldable component are in a shape of at least one of ovoid arch, tapered arch, or square arch, to conform to a curvature of the user's upper dental arch and the user's lower dental arch, respectively.

20. The oral appliance of claim 17, wherein the upper frame and the lower frame have at least one inner wall and at least one outer wall, and a distance between the at least one inner wall and the at least one outer wall is between 0.3 mm to 8 mm.
